(19) United States
(12) Patent Application Publication
(10) Pub. No.: US 2009/0043230 A1
(43) Pub. Date: Feb. 12, 2009

(54) BIOMECHANICAL MONITORING APPARATUS

(75) Inventors: John Ronald Davis-Havill, Hamilton (NZ); John Leonard Walley, Christchurch (NZ)

Correspondence Address: Davidson, Davidson & Kappel, LLC, 485 7th Avenue, 14th Floor, New York, NY, 10018, US

(73) Assignee: MOVEMENT METRICS LIMITED, Hamilton (NZ)

(21) Appl. No.: 11/629,001

(22) PCT Filed: Jun. 9, 2005
(86) PCT No.: PCT/NZ2005/000124

§ 371 Date: Sep. 22, 2008

(30) Foreign Application Priority Data
Jun. 10, 2004 (NZ) 533460

(51) Int. Cl. A61B 5/103

(52) U.S. Cl. 600/595

(57) ABSTRACT

The present invention relates to a biomechanical monitoring apparatus (1) which includes a mounting means adapted to allow the apparatus to be worn by a user and a motion a detection means (2) adapted to detect the motion of a user when the apparatus is worn. The apparatus also includes an accumulation means (4) adapted to indicate the extent of motion activity completed by a user since a specific time. The invention also relates to a method of using such apparatus where the extent of motion activity completed is compared against at least one movement activity threshold level to potentially activate an alarm indicator when such thresholds are exceeded.

### TECHNICAL FIELD

This invention relates to a biomechanical monitoring apparatus. Preferably the present invention may be adapted to be worn by users to provide an indication of the extent of prior activity of the wearer over a specific period of time. Preferably, the apparatus may provide an accumulative activity indication where activity is measured over a plurality of distinct and separate movements of a wearer or user.

Reference throughout this specification will also be made to the present invention being used in this particular configuration but those skilled in the art should appreciate that other applications are also envisioned.

### BACKGROUND ART

Many different types of biological mechanical motion monitors (or biomechanical monitors) have been developed. These types of apparatus are used to track the extent of specific activities or single movements executed by a user. Normally these types of apparatus are implemented through some form of sensor, sensors or apparatus worn on the body of the person executing the movements being monitored.

A good example of these types of apparatus can be found described in European patent specification No. EP 0336030, which describes a lifting monitor and exercise training system. The device employed is adapted to be worn by the person to be monitored as they execute a number of movements. The outputs of sensors integrated into the device are monitored to in turn provide a measurement of the activity completed by the wearer.

However, these types of apparatus only provide a limited amount of information with respect to distinct or specific movements or activities of a user. Data relating to particular measurements made over a single movement can be obtained using such devices, but these existing systems do not provide the facility for monitoring to occur over relatively long periods of time or over several or multiple instances of movements or loads.

Research into the causes of muscular aches and pains, joint damage and in particular into the causes of back pain have shown that damage can occur through the execution of a large number of small low intensity movements over an extended period of time—as opposed to just a single high intensity movement. Therefore, monitoring such low intensity movements over an accumulative or relatively long period would be an advantage in assessing the potential for damage or pain occurring.

In particular back pain is a common complaint where it would be preferable to monitor the activity of sufferers over time. Problems with a patient's lower back can be quite complicated due to subtle interactions of movements or displacements between the lumber spine and the pelvis. Displacements occurring in these two regions can in some instances be in the same direction while in others may diverge from one another dependent on the type of condition suffered by a patient and also the posture of the patient at the time. Those skilled in the art should appreciate that these factors make back pain complaints relatively complicated to monitor and treat.

It would be preferable to have an accumulative biomechanical monitoring apparatus which addressed or all of the above problems. Specifically, an apparatus which could be adapted to be worn by a user and which monitored the activities of a user over multiple and distinct movements in an accumulative manner, and which preferably could also provide an indication or warning once a specific activity threshold has been exceeded by a wearer would be of advantage.

Furthermore, it would also be preferable to have an accumulative biomechanical monitoring apparatus which was capable of inferring or indicating the motions executed by both the spine and pelvis of a wearer to assist in the treatment of a back pain ailment. Furthermore, a monitoring apparatus which could assist in the development of a treatment plan for back pain and which could monitor the effectiveness over time of such a treatment plan would also be of advantage.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

It is an object of the present invention to address the foregoing problems or at least to provide the public with a useful choice.

Further aspects and advantages of the present invention will become apparent from the ensuing description which is given by way of example only.

### DISCLOSURE OF INVENTION

According to one aspect of the present invention there is provided a biomechanical monitoring apparatus which includes,

a mounting means adapted to allow the apparatus to be worn by a user, and
a motion detection means adapted to detect motion of a user when the apparatus is worn by said user, and
an accumulation means adapted to indicate the extent of motion activity completed by a wearer since a specific time.

According to a further aspect of the present invention there is provided a biomechanical monitoring apparatus substantially as described above wherein the motion detection means is adapted to detect at least one specific type of motion of a wearer.

According to a further aspect of the present invention there is provided a biomechanical monitoring apparatus substantially as described above which includes an indicator means adapted to signal when a threshold activity level has been exceeded by a user wearing said monitoring apparatus.

According to a further aspect of the present invention there is provided a biomechanical monitoring apparatus substantially as described above which includes a trigger means adapted to control the degree of motion which will cause activation of the accumulation means.

According to yet another aspect of the present invention there is provided a method of using a biomechanical monitoring apparatus substantially as described above characterised by the steps of:
(i) mounting the biomechanical apparatus on a user to allow the user to wear said apparatus, and
(ii) detecting at least one specific type of motion of the user wearing the apparatus, and
(iii) activating the accumulation means of the apparatus to provide an accumulative indication of the extent of motion activity completed by the wearer since a specific time, and
(iv) repeating step (iii) as above for each subsequent motion of the user detected.

According to yet another aspect of the present invention there is provided a method of using a biomechanical monitoring apparatus substantially as described above characterised by the steps of:
(i) mounting the biomechanical apparatus on a user to allow the user to wear said apparatus, and
(ii) detecting at least one specific type of motion of the user wearing the apparatus, and
(iii) activating the accumulation means of the apparatus to provide an accumulative indication of the extent of motion activity completed by the wearer since a specific time, and
(iv) processing the accumulative indication of the extent of motion activity supplied by the accumulation means using a processing means, and
(v) repeating steps (iii) through (iv) as above for each subsequent motion of the user detected.

According to a further aspect of the present invention there is provided a method of using a biomechanical monitoring apparatus substantially as described above further characterised by the additional subsequent step of activating an indicator means to signal when a threshold activity level has been exceeded by the user wearing the apparatus.

According to yet another aspect of the present invention there is provided a method of using a biomechanical monitoring apparatus substantially as described above wherein the apparatus is mounted on a user above the user's pelvis and below the user's lowest rib at the thorax.

According to a further aspect of the present invention there is provided a method of using a biomechanical monitoring apparatus substantially as described above wherein the apparatus is mounted on a user below the end of the user's twelfth rib and at substantially the middle of the iliac crest of the ilium.

According to another aspect of the present invention there is provided a method of using a biomechanical monitoring apparatus substantially as described above, characterised by the steps of;
i) mounting the biomechanical monitoring apparatus on a user, and
ii) initialising the biomechanical monitoring apparatus when the user holds an initialisation stance, and
iii) setting at least one threshold activity level for said user.

According to yet another aspect of the present invention there is provided a method of using a biomechanical monitoring apparatus substantially as described above, characterised by the additional subsequent step of;
iv) determining the orientation of the biomechanical monitoring apparatus when mounted on the user.

According to yet another aspect of the present invention there is provided a method of using a biomechanical monitoring apparatus substantially as described above wherein a threshold activity level is set during the user executing at least one hazardous motion.

According to a further aspect of the invention there is provided a method of using a biomechanical monitoring apparatus substantially as described above wherein a threshold activity level is set at a position within the user executing a hazardous motion at which the user first experiences pain.

According to yet another aspect of the present invention there is provided a method of using a biomechanical monitoring apparatus substantially as described above wherein previous accumulative indications of the extent of motion activity of the user are used to subsequently update at least one threshold activity level for said user.

Preferably the present invention is adapted to provide a biomechanical monitoring apparatus. Such an apparatus may be used to track the movement activity of a person or body, preferably over an extended period of time. Biomechanics and biomechanical monitoring apparatus are relatively well known in the art but existing systems have a number of limitations with respect to the extent of time over which monitoring can occur, and also the number of specific movement of a user or wearer which can be investigated over a period of time.

Preferably the apparatus provided in accordance with the present invention may be adapted to monitor biomechanical movements of a user or wearer and provide an accumulative indication of the extent of the group of such movements.

Reference throughout this specification will also be made to the present invention incorporating an accumulation means adapted to indicate the extent of motion activity completed by a wearer since a specific time. The extent of motion activity indicated may encompass both motion detected in relation to a user, in addition to a mass, weight or loading experienced by a user when moving. Motion activity may then be defined as the combination of both a specific motion of a specific mass/load.

In a further preferred embodiment, the present invention may be adapted to monitor movements or body activity involving the bending of a user's spine or back. Back pain is a very common ailment which is generally caused or aggravated by a sufferer overusing their back through a large number of small low intensity movements executed over an extended period of time. Back pain may also be aggravated through loading of the spine caused by the weight present in both the user's body and any additional loads, or weights moved by the user in a back related motion.

Reference throughout this specification will also be made to the present invention being employed to monitor body motions and loads associated with the back or bending of the spine. However, those skilled in the art should appreciate that other types of movements and specific activities related to other areas of the body or different joints can also be monitored in conjunction with the present invention.

In a preferred embodiment the monitoring apparatus may include a mounting means. Such a mounting means may be adapted to allow the apparatus to be worn on the body of a user, preferably allowing the apparatus to be attached to the clothing of the wearer in the region or vicinity of the wearer's body at which motion measured closely represents or can be used to infer the biomechanical movements that are the target of the measurement and monitoring.

In one alternative embodiment, the mounting means may consist of or include a clip type mechanism allowing the apparatus to be clipped to the waist or hip region of a wearer's clothing. For example, in a preferred embodiment, a clip may be provided as a mounting means to allow the apparatus to be clipped to a belt worn by a user.

However, in a preferred embodiment a mounting means may be formed by a belt adapted to encircle and engage with the body of a user. The main body of the monitoring apparatus may be connected to this belt and the belt may be preferably tightened around the body of a user above the user's pelvis and below the lowest rib at the thorax of the user.

In a further preferred embodiment the belt based mounting means provided may be used to attach the monitoring apparatus to a user below the end of the twelfth rib of the user and the mid point of the iliac crest of the ilium. This position of the monitoring apparatus has being developed specifically for the monitoring of the activity of users which suffer from back pain. At this position movement of the monitoring apparatus can be used to infer the motion of both the pelvis and lumber region of the user's spine, allowing both bending and twisting motions to be monitored concurrently in conjunction with the present invention. At this position a single piece of apparatus may be employed to infer or determine the motions of both a user's pelvis and lumber spine region.

Reference throughout this specification will also be made to the mounting means being provided through a belt which allows the apparatus to be worn above the pelvis and below the lowest rib at the thorax. However, those skilled in the art should appreciate that other types of mounting means such as for example, Velcro patch attachments, tie strings, or integration of the apparatus into the fabric of a garment may also be employed in conjunction with the present invention, and reference to the above only throughout this specification should in no way be seen as limiting.

Preferably the present invention may be employed by a patient or sufferer of pack pain and also a physician or clinician tasked with treating the patient and preparing a treatment plan to improve the patient's condition. The present invention may be used by both parties to provide an accumulative indication of the user or patient's activity, preferably relative to one or more threshold activity levels set by a physician or clinician. Furthermore, the present invention may also be employed to record and present data indicative of a user or patient's prior activities with a view to assessing both the effectiveness of the current treatment plan prescribed and to assist in resetting or updating at least one threshold activity level employed in conjunction with the present invention.

Reference throughout this specification will also be made to a user of the present invention being the wearer of the device who executes or completes the motion activities monitored. However, those skilled in the art should appreciate that other users such as for example managers, physicians, clinicians or caregivers for the wearer may also use the present invention.

In a preferred embodiment, the monitoring apparatus may include a motion detection means. A motion detection means may be adapted to detect the motion of a user when the apparatus provided is worn by such a user.

In a further preferred embodiment, the detection means employed may be adapted to detect one or more specific types of motion only of a wearer. For example, in a preferred embodiment where the monitoring apparatus employed to monitor the back movements of a wearer, the detection means may only detect movement specific to the hips, spine or back of the wearer. In this configuration of the present invention, particular movements of the wearer may be targeted and monitored only for loads, specific body parts or types of motion of concern to the user or their physician, clinician or other third party.

Reference throughout this specification will also be made to the motion detection means primarily detecting only motions related to a wearer's hip rotation, side flexion or forward bending at the waist. However, those skilled in the art should appreciate that other different types of specific motions or movements may also be monitored in conjunction with the present invention and reference to the above only throughout this specification should in no way be seen as limiting.

In some alternative embodiments mercury based tilt switches with associated ancillary support electronics may be employed as part of a motion detection means. These types of switches may provide an electrical trigger signal when tilted or moved. In yet other alternative embodiments piezo electrical materials may also be employed as part of a motion detection means to provide an electrical signal on detection of motion or pivoting of a housing incorporating the present invention.

However, in a preferred embodiment the motion detector employed may be formed from an accelerometer or a plurality of accelerometers. For example, in some instances solid state silicon chips may be used which incorporate such an accelerometer or accelerometers, in addition with other ancillary or support circuitry. Such “single chip” solutions may provide a cost effective and compact implementation for the motion detector required.

Reference throughout this specification will however be made to the motion detection means being formed from or incorporating a pair of accelerometers embedded within a single integrated circuit chip. Furthermore, specific references will also be made to the present invention employing the ADXL311 chip from Analogue Devices and as discussed in analogue devices website, www.analogue.com. However, those skilled in the art should appreciate that other configurations of the motion detection means required may be employed and reference to the above only throughout this specification should in no way be seen as limiting.

In such a preferred embodiment, the pair of accelerometers employed may provide or have their outputs sampled to produce both positive and negative sensor readings from each accelerometer. Positive and negative sensor values may be obtained from movements in opposite directions giving, for example in a preferred embodiment, positive and negative X axis movement readings, and positive and negative Y axis readings. Readings from a single accelerometer in a positive or negative direction may preferably be treated separately so as to ensure that physical activity in both directions does not necessarily cancel out the accumulative record of activity to be maintained.

Preferably the monitoring apparatus may include an accumulation means which is adapted to indicate the extent of motion activity completed by a wearer since a specific time. The accumulation means may in some instances be capable of selective activation so that when a specific motion of a wearer is detected the accumulation means will then be activated to increase a record or count the extent of the activity of the wearer.

In some embodiments, the accumulation means may provide an incremental measurement facility so that detected motion of a user will be used to activate components employed in the accumulation means to increment the record maintained.

Furthermore, in additional embodiments the accumulation means may also be linked or tied into any mass determination means provided. In these instances, a combination of both detected motion and the load or mass moved by a user can be employed to allow the accumulation means to indicate the extent of motion activity completed.

Those skilled in the art should also appreciate the accumulation means may record or indicate an extent of activity composed of a plurality of separate movements over a relatively long period of time as opposed to the intensity of one single movement. Furthermore, the accumulation means need not necessarily provide a visual or audible indication to a user in some instances, but may simply maintain a record of prior motion activity since a specific time.

In some embodiments the accumulation means may be formed from a clockwork mechanism or time keeping electrical circuitry which can provide an accumulative or incremental record of time expired once such systems have been activated. Preferably activation of such timing systems by the motion detection means will be maintained until the motion detection means no longer indicates the user or wearer of the invention is performing a specific motion or action. In this way the timer provided as an accumulation means can provide an accumulative record of the amount of time the wearer has been performing a particular movement where this time record is cumulative over many separate movements.

In alternative embodiments, such timer based systems, mechanisms or electronic circuitry may not necessarily be provided or used as an accumulation means in accordance with the present invention. For example, in one alternative embodiment, simple latch based electrical counter components may be provided as an accumulation means where the latch is activated each time that the user performs one of the motions to be monitored, irrespective of the duration over which this motion occurs.

However, in a preferred embodiment an accumulation means may be formed from a microprocessor with ancillary memory elements where this microprocessor receives at least one input signal from the motion detection means and/or mass determination means. Such a microprocessor and associated memory may record, process or calculate any particular values or components of interest—such as a number of times the wearer starts the movement involved, the duration of each movement, the times of the day in which the movements were completed, the frequency of movement or any rest periods between movements.

In a further preferred embodiment, such a microprocessor may be programmed or loaded with software adapted to execute processing algorithms on input signals received. Relatively sophisticated software and processing algorithms may be employed to provide an extent of motion activity indication which can take into account both the motions completed by a user in addition to the load present on the user during motion.

Reference throughout this specification will however in general be made to a microprocessor forming an accumulation means which processes motion detection signals in isolation.

In embodiments which employ a microprocessor and at least one memory element in combination with the accelerometer based movement sensors discussed above, separate accumulative records of positive X, positive Y, negative X and negative Y direction movements may all be maintained independently by the microprocessor and associated memory elements. This allows independent processing and analysis of each specific movement in a single direction at a later date, or in real time if required.

Furthermore, in some embodiments additional processing of such input accelerometer values may also be completed to provide a single composite value indicative of the user's overall cumulative activity level. In such instances, the activity level value calculated may be provided through a function which uses the output of both accelerometers as its input. Such algorithms or functions may be adapted to provide output parameters related to specific movement issues of a wearer, where these output parameters may in turn be used to configure or operate an indicator means, or may be displayed to a user or wearer of the present invention. The particular type of function or algorithm employed in such instances will depend on their specific movement of interest, and also potentially, any specific health concerns of a wearer or user of the present invention.

Reference throughout this specification will also be made to the accumulation means being provided through a microprocessor and memory element combination. However, those skilled in the art should appreciate that other configurations and implementations of an accumulation means may be provided and reference to the above only throughout this specification should in no way be seen as limiting.

In a preferred embodiment the accumulation means may sample the output signal or signals obtained from the motion detection means. Sampling of signals allows a series of discreet, instantaneous measurements to be made at the sampling frequency employed depending on the power or speed of operation of the accumulation means used. For example, in some embodiments of the present invention, a sampling frequency of between 1 to 200 Hertz may be employed, and in some applications where a high resolution of measurements is required, a high sampling frequency of (for example) 100 Hertz may be used to capture a significant amount of data over a relatively short period of time.

In a preferred embodiment an initialisation or set up process may be executed in relation to the monitoring apparatus provided when this apparatus is initially mounted on a user. In such instances a user may be directed to hold at least one known initialisation stance concurrent with the output signal or signals of the motion detection means being recorded. The value or level of the output of the accumulation means may therefore be associated with a zero or baseline level measurement when the user holds or exhibits the initialisation stance involved. For example, in one preferred embodiment an initialisation process may be executed when a user stands upright, without bending over or twisting their torso. The output of the motion detection means at this point will have been set to a zero baseline level measurement.

In a further preferred embodiment, the present invention may also incorporate or include an indicator means. Such an indicator means may be formed from a type of element which can provide a signal to a user or wearer of the present invention. Such signals may be used to indicate to a wearer that a particular threshold activity level assigned or associated with the wearer has been exceeded. One or more particular or selected threshold activity levels may be assigned, for example, to each wearer depending on their physical fitness, condition and previous medical history. However, those skilled in the art should also appreciate that in other alternative embodiments, the indicator means may be operated or triggered independent of the activity level of a user or wearer. For example, in such embodiments the indicator means may be triggered at a specific time of day or after a specific period of time—independent of the accumulative activity of the wearer if required.

In a preferred embodiment the present invention may be used to monitor a user's activity in association with one or more threshold activity levels assigned for the user. A threshold activity level may in some instances consist of a value for an accumulative record of prior activity completed by a user which may be exceeded by further subsequent activity. However, in other instances a threshold activity level may consist of an angle or degree of bodily extension, twisting or bending which a user should not exceed. Furthermore, in other instances a threshold activity level may be composed of both an indication of an angle or degree of bending or twisting in combination with a time duration indication or limit associated with such a motion. In such instances a threshold activity level may be exceeded if a user moves past a particular degree of bodily extension for more than a set time duration limit. Furthermore, in other instances a threshold activity level may be associated with a recovery time for a user, where this threshold will be exceeded if after a particular activity with a specific degree of extension, the user completes a further activity with a set degree of extension prior to the expiry of a recovery time.

In some embodiments the present invention may be used to provide an accumulative monitor or indication of activity which is compared against at least one threshold activity level which may be employed to give a warning signal or indication when this threshold level has been exceeded or breached. In this way the present invention may be provided to warn a wearer that they are overusing a particular movement and should potentially stop their activities and rest, or perform any number of recuperative or stretching exercises.

Furthermore, the present invention may be employed in some embodiments to monitor the activity level of a user and to compare same against one or more threshold activity levels, where the information recorded may subsequently be reviewed by a physician or clinician treating the user with a view to implementing a treatment plan for the user. In such instances, accumulated information or data associated with the user's activities may be employed to develop and refine a treatment plan for the user in addition to monitoring to the recuperation of the user over time.

In some embodiments, an indication means may consist of a buzzer or bell mechanism which can provide an audible warning indication signal to a wearer. This bell or buzzer may be triggered once the threshold activity level assigned for a particular wearer has been exceeded.

However, in alternative embodiments the indicator means may consist of a warning light which can be illuminated to flash on and off again to attract the wearer's attention and provide a warning signal indicating that a user's activity threshold level has been exceeded.

In a preferred embodiment, the present invention may also include an adjustment facility whereby a particular wearer's threshold activity level or levels may be amended or changed. For example, in embodiments where a timer system is employed in conjunction with an accumulation means, the time allotted to a particular wearer may be increased or decreased to in turn change the threshold activity level assigned to the user. In other embodiments where the invention employs a microprocessor and associated memory elements as an accumulation means, the programming of the microprocessor may be adjusted or modified to in turn change a threshold activity level assigned to a user. Such an adjustment facility may allow a threshold level assigned to a user to be varied depending on the current condition of the user or wearer.

In a preferred embodiment a threshold activity level may be set during a user executing at least one hazardous motion. A hazardous motion may be defined as a motion which has the potential to aggravate a complaint or condition of a user which is to be monitored in conjunction with the present invention.

In a preferred embodiment a threshold activity level may be set at a position within a hazardous motion in which the user first experiences pain. The extent of a hazardous motion which a user may execute without experiencing pain may therefore be identified. This approach can determine the extent of the user's ailment and tailor the setting of threshold activity level to suit same.

In some embodiments prior records of accumulative indications of the extent of motion activity of a user may also be used to assist in the setting of at least one threshold activity level for this user. For example, in some embodiments an initial threshold level may be set, and then subsequently updated depending on a record of the user's previous accumulated activity as recorded in conjunction with the present invention. This previous activity record may preferably provide a treating physician or clinician with a clearer picture as to how a patient is recuperating under a treatment plan prescribed by the physician or clinician.

Preferably, the present invention may also include a user operable threshold setting element, such as for example, a button on an exterior housing or casing of the monitoring apparatus. This element may be actuated, preferably during the execution of a hazardous motion by a user, to set a threshold level indicative of an absolute angle or degree of bodily extension, twisting or bending experienced during the execution of the hazardous motion involved.

Preferably, the present invention may be used within an iterative treatment process which allows for an initial set of one or more threshold activity levels to be set by a physician or clinician and then subsequently updated or modified over time as the patient follows a treatment plan. The threshold activity level or levels set may therefore track the progress of healing or recuperation of a user over time and may assist a physician or clinician in assessing the effectiveness of the treatment plan prescribed for their patient. Furthermore the accumulative record of activity provided in conjunction with the present invention may also be employed by a physician or clinician to again assist in the tailoring or modification of any treatment plan prescribed for a patient.

In a further preferred embodiment, a microprocessor used to form at least a portion of the accumulation means may be loaded with instructions or software which allowed for the automatic calibration of the output received from the motion detector. In such instances where a user first mounts or puts the present invention on their body, such auto-calibration procedures may be triggered to determine the current orientation or positioning of the monitoring apparatus as the wearer executes at least one hazardous motion employed as discussed above. The hazardous motion executed may have a predefined direction which will in turn allow the signal received from the motion detection means to be used to determine the orientation of the monitoring apparatus when mounted on the user.

For example, such a process may be used to determine whether the monitoring apparatus is mounted on the left hand or right hand side of a user's body depending on whether a particular motion detection signal received is positive or negative during the execution of the hazardous motion involved. This process may allow the microprocessor to assign specific X Y axis orientations to measurements obtained to in turn determine at a later date the type and extent of movements subsequently completed by the wearer.

In some alternative embodiments, the present invention may also include a trigger means which is adapted to control the degree and/or duration of motion of a user which will activate the accumulation means. Such a trigger means can be used to adjust the degree, duration or depth of movement which will in turn be detected by the detection means and in turn activate the accumulation means. Such a trigger means may be used to adjust the behavioural mechanisms of the present invention depending on the particular user involved and their current physical condition.

Those skilled in the art should appreciate however that such a trigger means need not be considered essential to the operation of the present invention in some embodiments. For example, a case where the accumulation means is implemented through a microprocessor and associated memory, all signals received from a motion detection means may be recorded with out any need to trigger such recording processes depending on the degree or duration of movement of the user.

In a further preferred embodiment, a basic record of accumulative activity or incremental count indicative of motion activity maintained by an accumulation means may be reset periodically, either automatically or by a user. The time at which the accumulation means is then reset will provide the specific time over which a current accumulative indication of activity has occurred. Furthermore, in other embodiments an incremental record of motions detected in relation to a user over time may also be reset periodically in substantially the same manner as discussed above.

In some alternative instances a record or an incremental count provided by the accumulation means may be reset once an indication means (such as a buzzer or light) has been activated to warn a wearer. At this stage the wearer should be aware that they should rest or perform recuperative or conditioning exercises such as stretches or other similar movements, potentially to prevent damage occurring to the region or body part associated with the movement which triggered the indicator means. Once the indicator has been activated and the wearer has taken the appropriate rest of recuperative action, a count or record maintained by the accumulation means may then be reset to zero.

In a preferred embodiment where the accumulation means is formed from or incorporates a microprocessor and ancillary memory elements, the present invention may also be adapted to provide or upload a logged record of motion activity to a remote computer system. Such a log of activity may provide a significant amount of data with respect to the activity level of a wearer over a relatively long period of time. For example, such information may provide a profile of the duration of movements executed, including their degree and extent and also the times of day at which particular movements where executed and their frequency at such times of day.

For example, in a preferred embodiment where a pair of accelerometers are used as the motion detector, a log or record of positive and negative accelerometer value measurements for each accelerometer may be recorded and subsequently be provided and an upload to a remote computer system. Furthermore, in other configurations, such records of accelerometer readings may in turn be pre-processed to provide a single or overall activity measurement again to subsequently be provided in an upload to a remote computer system.

However, those skilled in the art should appreciate that this facility need not be considered essential to the present invention as the simple provision of an indicator bell or buzzer may provide advantages for a basic user.

The present invention may provide many potential advantages over the prior art.

The present invention may give an effective warning to a user that they have exceeded a specific accumulatively based activity threshold level by completing a plurality of separate and distinct movements. This accumulative indication of activity can be used to monitor the extent and frequency of a large number of individual movements, which when accumulated together, have the potential to cause injury or pain to the person being monitored.

Furthermore, with the provision of a mass determination means the extent of activity may be measured from both the motions completed by a user. In such instances, the present invention may provide an accurate and useful indication of the extent of a user's activity.

Furthermore the present invention may also provide the facility to have various operational behaviours or parameters adjusted depending on the physical condition of the current user employing the monitor apparatus.

The present invention may also in some instances be constructed from simple mechanical components which are relatively expensive to manufacture and maintain. In other cases, the present invention may be extended to include relatively sophisticated electronic components which can, if required, provide highly detailed information with respect to the profile of a user's activities over an extended period of time.

Furthermore, the present invention may also infer measurements of the motion of both a user's pelvis and lumber spine region through the provision of a single motion detection means mounted above the pelvis and below the user's lowest rib at the thorax. This particular mounting position allows movement of both sections of the body to be monitored with the main body of the monitoring apparatus being located at a single position.

Furthermore, the present invention may also facilitate the development and assessment of a treatment plan provided by a physician or clinician for a patient. A physician or clinician may set one or more threshold activity levels for the patient and update these threshold levels over time depending on how the patient's recuperation proceeds, and also in some instances through a review of the accumulative record of a patient's activity provided in conjunction with the present invention.

### BRIEF DESCRIPTION OF DRAWINGS

Further aspects of the present invention will become apparent from the following description which is given by way of example only and with reference to the accompanying drawings in which:

FIG. 1 illustrates a block schematic diagram of components employed to form a monitoring apparatus in one embodiment, and

FIG. 2 shows an information flow schematic for data generated using the monitoring apparatus discussed with respect to FIG. 1, and

FIG. 3 illustrates a block schematic flow chart of a process executed to initialise and use a monitoring apparatus provided in accordance with the present invention, and

FIG. 4 shows a screen shot of an interface software system adapted to configure the movement apparatus discussed with respect to FIG. 3, and

FIG. 5 provides a graphical illustration of a number of threshold activity levels set in accordance with the embodiment discussed with respect to FIGS. 3 and 4, and

FIG. 6 shows a number of report graphs which may be displayed in conjunction with the present invention.

### BEST MODES FOR CARRYING OUT THE INVENTION

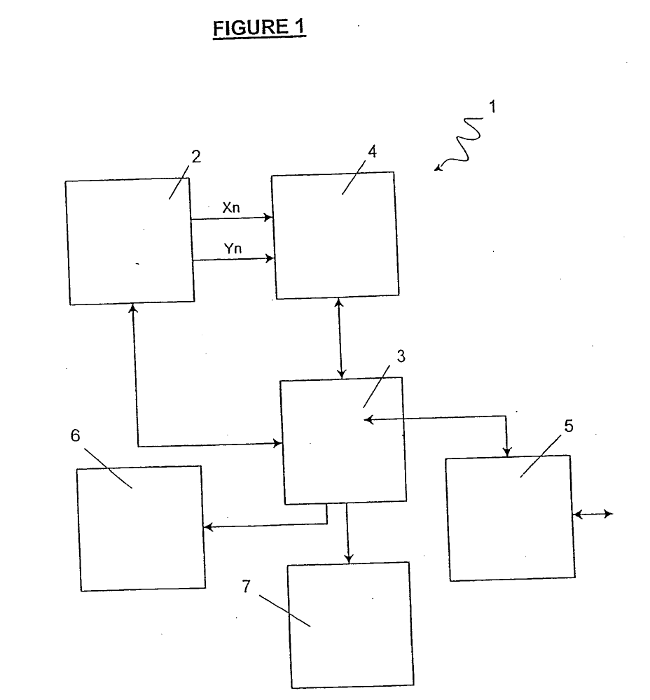

FIG. 1 illustrates a block schematic diagram of components employed to form a monitoring apparatus in one embodiment.

The monitoring apparatus (1) includes in the embodiment shown a motion detection means, formed by a pair of accelerometers integrated into a single integrated circuit chip (2). In a preferred embodiment of the present invention this chip is marketed by Analogue Devices under Part No. ADXL311. The ADXL311 includes two independent accelerometers which provide motion detection in two planes of motion, as indicated by the Xn, Yn output lines shown with respect to FIG. 1.

The monitoring apparatus also includes an accumulation means, which is implemented in the embodiment shown through the combination of a microprocessor (3) and associated memory element in the form of an EPROM (4). Both the EPROM memory (4) and microprocessor (3) can receive signals from the motion detector accelerometers (2) and can record a cumulative measure of activity of a person wearing the monitoring apparatus.

The monitoring apparatus (1) also includes communications circuitry and a connector (5) which is used to implement a computer based serial communications port. This port can interface the monitoring apparatus with a remote computer system and allow the contents of the memory EPROM (4) to be uploaded and supplied to such computer systems. Furthermore, the processor (3) may also be used to further process the raw data received from the accelerometers (2) and to in turn store a combined activity value in calculating the result of a function which receives both the X and Y direction movement values detected by each accelerometer.

The monitoring apparatus (1) also includes an indicator (6) directly linked to the processor. In a preferred embodiment, such an indicator is formed from an audio based alarm system which can be activated by the processor when a specific continuous threshold of activity has been exceeded by a wearer or user of the monitoring apparatus.

The monitoring apparatus (1) also includes a reset or on/off switch (7) again linked to the microprocessor (3). This switch allows the wearer to switch the monitoring apparatus on or off as required and also to reset the monitoring apparatus accumulative record of activity if required.

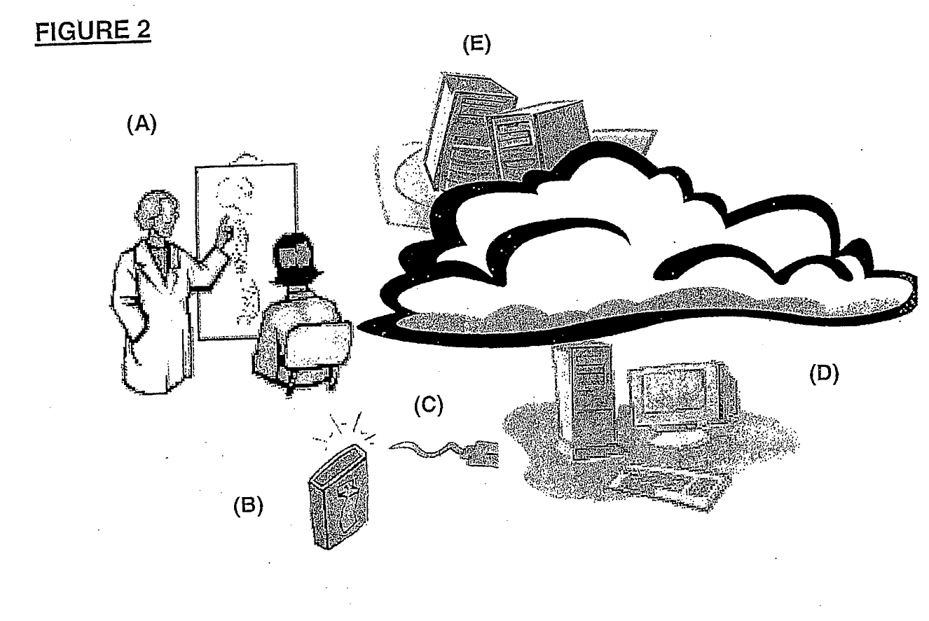

FIG. 2 shows an information flow schematic for data generated using the monitoring apparatus discussed with respect to FIG. 1.

In the steps (A) through (E) shown, the biomechanical monitoring apparatus discussed with respect to FIG. 1 is employed to generate data or information to be used in a medical analysis process.

Initially a user of the apparatus consults a physician or clinician or medical practitioner to discuss a particular ailment affecting the user. Next at step (B), the monitoring apparatus shown is worn by a user throughout their daily regime. A mounting means, shown in FIG. 2 as a belt hook clip, is used in the embodiment shown to allow the apparatus to be worn by a user.

At step (C) of this process, the contents of the memory of the monitoring apparatus is uploaded through a serial connection to a remote computer system, preferably owned and operated by the wearer or user of the monitoring apparatus.

At step (D) the data or information uploaded is subsequently transmitted across a further computer network (or potentially the internet) to a remote server manager operated by a medical organisation or a medical service provider. Such a remote server may record the data generated or stored by the apparatus over time and potentially can be used for further processing work to suggest treatments for the user involved, or alternatively new operational parameters for the monitoring apparatus.

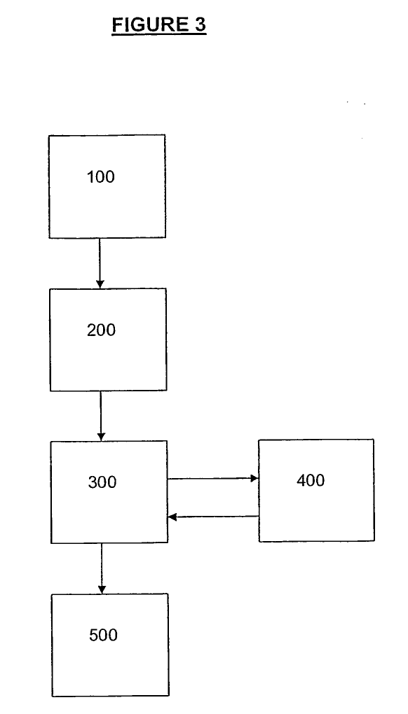

FIG. 3 illustrates a block schematic flow chart of a process executed to initialise and use a monitoring apparatus provided in accordance with the present invention.

In the set up and initialisation process illustrated the first stage (100) of this process is completed when the monitoring apparatus is mounted to the body of the user. Preferably the apparatus is mounted above the user's pelvis and below the user's lowest rib. This mounting position allows motion of both the user's lumber spine and pelvis to be inferred from the placement of a single apparatus.

Next, an initialisation or zeroing process is completed at stage 200 where the user is asked to hold an initialisation stance while the output of a motion detection means incorporated within the monitoring apparatus is recorded. Preferably this initialisation stance may be implemented by a user standing upright without bending forward or twisting sideways to give a zero level of activity against which the normal output of the motion detection means may be compared.

The next stage of this initialisation and set up process is shown as steps 300 and 400 where a number of threshold activity levels are set for the user who is to wear the monitoring apparatus. The setting of threshold activity levels is preferably an iterative, consecutive process which is completed over time, and shown by stage 400, through the user completing one or more hazardous motions in combination with a comparison of previous activity indication data recorded by the monitoring apparatus. For example, in a preferred embodiment a user may be asked to bend over and twist to one side to complete a hazardous motion where a threshold activity level will be set at the point in this motion at which the user first feels pain. Furthermore, the prior activity information supplied at stage 400 may assist a physician or clinician instructing the wearing user in the type of hazardous motion to be completed when setting a threshold.

This set up and initialisation process is then completed at stage 500 shown where the orientation of the monitoring apparatus when mounted on a user is determined. Through the execution of the hazardous motion discussed above the output of the motion detection means is analysed to determine what side of the body the monitoring apparatus is mounted on. This orientation information may then be recorded in conjunction with the monitoring apparatus to allow effective analysis of accumulative activity information recorded in relation to same at a later date.

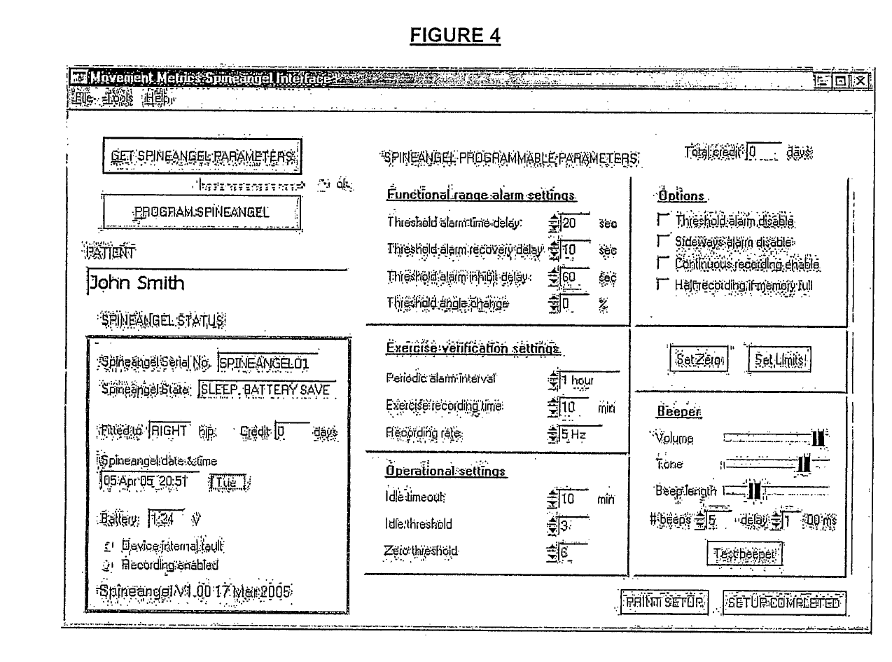

FIG. 4 shows a screen shot of an interface software system adapted to configure the movement apparatus discussed with respect to FIG. 3.

As can be appreciated by those skilled in the art the majority of the settings illustrated in FIG. 4 are relatively self explanatory. The operation of the monitoring apparatus may be modified through such an interface to, for example, adjust the volume, tone and time period of an audio based alarm indicator.

This interface screen also allows a physician or clinician to modify parameters associated with the operation of the monitoring apparatus such as the parameters shown under the functional range alarm settings.

For example, a physician or clinician may delay the activation of an alarm indicator by the 20 seconds shown and may also set the alarm to go off again immediately if a wearer does not rest more than 10 seconds prior to completing a further movement. The activation of an alarm may also be inhibited by 60 seconds as shown, and the physician or clinician may also set an absolute threshold angle change which, if detected, will automatically trigger an alarm.

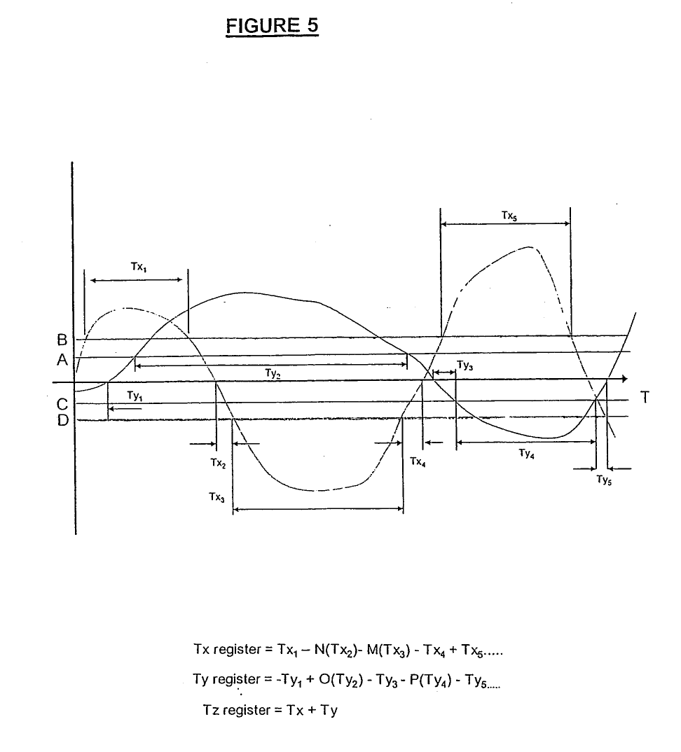

FIG. 5 provides a graphical illustration of a number of threshold activity levels set in accordance with the embodiment discussed with respect to FIGS. 3 and 4.

Levels A and C are preset activity level thresholds associated with movement in the y plane, whereas levels B and D are similar limits associated with movement in the x plane. The coefficients n, m, o and p are again predefined, preferably by a physician or clinician. Accumulative records of motion activity in the x and y plane will also be recorded in the Tx register and the Ty register using the formulas shown. An overall accumulative motion activity value is also recorded in the Tz register, being equal to the combination of the Tx and Ty registers.

In the instance shown both movement extent and time duration information is employed to set a threshold activity level. For example, as can be seen from FIG. 5, level B is exceeded by period tx₁ and tx₅. An activity level threshold may be set so that an alarm will be triggered after the breaching of threshold level in addition to the user exceeding a particular duration time for a movement which exceeds this level.

These aspects of the operation of the present invention are discussed in more detail below with respect to the treatment of back pain relating to inter-vertebral disc origin, and back pain of facet join origin.

### Back Pain of Intervertebral Disc Origin—Including Annular Damage, Bulging Disc or Herniating Disc:

The “hazardous motion” for this healing tissue has been found in pathomechanical research to be bending and twisting away from the side of disc damage.

This setting allows full motion in all other directions to assist disc nutrition to facilitate healing.

The goal of these settings of the monitoring apparatus is to provide controlled functional loading in a way appropriate to the stage of the back disc problem, while providing dynamic education to the patient of functional biomechanics specific for their problem.

This is not only relevant to the particular episode of back pain but has a role in future prevention by biomechanical education.

Selecting of Location

The monitoring apparatus location used is on the side same side as the damaged region of the intervertebral disc. This is identified by several different methods that must all correlate for the conclusion of which side to locate the device.
a) The pain side
b) The side where there is the least side bend towards. This is found by measuring from the floor to the tips of the fingers when the patient has been instructed to bend sideways, to the left and the right.
c) The side where the pain was at the time of the injury.
d) The opposite side to the direction of twisting when the injury occurred eg bending and twisting to the left damages the right side, and bending and twisting to the right damages the left side.
e) The side of any identifiable pathology from MRI scan, or discography.

Positioning Device

The device is attached to a belt positioned above the centre point of the iliac crest and the twelfth rib on the specific side identified above.

Positioning the Patient

The patient is instructed to stand normally for the setting of the initialisation stance to determine a zero position.

Zeroing the Device

The device is zeroed by a sustained push on a threshold setting external button by either the clinician or the wearer. Following this the wearer maintains standing normally for a further 2 seconds until 2 beeps are heard, confirming the device position is committed to memory of the device.

Setting the Functional Movement Threshold Activity Level

The wearer is instructed to bend forwards and away from the device in a twisting motion and the threshold setting button is pushed once. This action also automatically sets the device orientation.

The patient is again instructed to perform the same movement forward and away from the device and the threshold button is pushed again, at a position decided on by the clinician setting the bending and side bending thresholds.

The decision as to what position the functional threshold is set at is dependant on several factors.

### Pain Intensity Behaviour During Movement

The patient is monitored to the pain intensity levels whilst moving into the motion of bending forwards and away from the device and the threshold is set when these pain level occur.

This is undertaken using the accepted proven reliable subjective pain rating scale the 0-10 numerical rating scale (NRS) where 0 is no pain and 10 is the worse pain imaginable.
a) At first onset of pain intensity level 1 NRS when performing the movement into the range of bending and twisting away from the device.
b) At an increase of pain intensity to 3 NRS when performing the above movement.
c) At an increase of pain intensity to 6 NRS when performing the above movement.
d) At an increase of pain to 9 NRS when performing the movement.

### Healing Time Since the Injury

The decision as to which pain level the device threshold is set at is a clinical decision based on the time passed since the original injury occurred.

How long it is since the injury occurred is related to the stability of the healing tissues and these time periods have been identified by the Quebec Task Force (Spitzer et al 1987).

Less than 7 days duration—Acute stage injury, so threshold is set earlier in the active range as soon as 1 NRS is achieved.

The pain level at the end of the movement when the threshold is set is increased on a graduated per consultation basis by increments of 1 NRS as long as the patients overall condition is improving.

7 days to 7 weeks duration—Subacute stage, injury more stable. The threshold is set at the point where 3 NRS is achieved and progressed in increments of 1 NRS per consultation

7 weeks or more duration—Chronic stage. In this phase the threshold is set at the end of the available movement range into the direction identified regardless of the pain level.

If the clinician gains the impression that the patient is not moving sufficiently for setting accurate thresholds, the clinician may direct a percentage increase in the threshold range to be loaded into the monitoring apparatus.

### Pain Location Moving

If the location of the pain begins to spread down the legs away from the back during the increasing range of motion then the patient should slowly return to upright without the threshold being set. The clinician then guides the patient into the movement again and sets the threshold at the first sign of the pain moving away from the back (peripheralising).

### Neurological Symptoms

The presence of neurological symptoms of pins and needles or numbness as the functional range increases is an indication to reduce the range of motion until these symptoms cease and set the threshold at that position.

Setting the Threshold Alarm Time Delay

This is set at a PC interface and recognizes the importance of sustained load on healing tissues and the problems that creep contribute to. Problems can occur if the load is sustained too long during the early healing phase or in any vulnerable disc tissue due to degenerative changes. The device allows this load to be increased in a graduated and controlled way and is set by increasing the time period at each consult.

The length of time the threshold can be exceeded for is dependant on the healing stage based on the definitions of acute, subacute and chronic as above and is also a clinician decision based on average daily pain intensity levels using the NRS.

Acute (less than a week duration)—5 seconds threshold alarm delay.

Increase the time period of threshold alarm time by 5 seconds each consult as long as average daily patient pain NRS is dropping.

Subacute (7 days to 7 weeks duration) Begin with 15 second alarm delay and progress by 10 seconds per consultation subject to improvement in average daily pain intensity NRS.

Chronic (7 weeks or more duration) rapidly increase the loading time before the alarm is sounded by 30 seconds per consultation.

Setting Recovery Period

The recovery period is set again via a PC interface and is the period of time that the patient must be inside (not exceeding) the threshold before they can exceed the threshold again without the alarm indicator ringing immediately.

This is in accordance with the pathomechanical principal of allowing time for reduction of hysteresis which makes tissues vulnerable to injury and contributes to more harmful effects of load.

This recovery time is set at each consultation in a progressively reducing way, so the tissues are give more time to recover unloaded in the early healing stage, and less time to recover as the tissues healing is more complete.

Acute Phase—(less than week duration). Recovery is set at 5 minutes.

Recovery period is reduced by 20 seconds per consultation.

Subacute Phase—(7 days-7 weeks) Recovery is set at 2 minutes, reducing by 20 seconds per consult.

Chronic Phase (beyond 7 weeks) Recovery phase normally set at a larger level than the subacute phase in recognition of the likely Discogenic degenerative changes contributing to the chronicity of the disorder.

### Back Pain of Facet Origin:

The hazardous motion for this type of back pain is an arching of the back in standing and walking. Typically this is slouched with the stomach protruding and the hips/pelvis region pushed forwards (excessive back arch or lordosis).

When in this standing and walking position, the facet joints of the back are crushed together causing a compression loading, contributing to pain and degenerative changes in these joints.

This is particularly relevant when such joint degenerative changes are already present.

This setting enables a method specific rehabilitation of the core trunk stabilizing muscles of lumbo-pelvic control which are involved in maintaining a reduced lumbar lordosis. (Reduced low back arch or lordosis)

Reducing the lumbar lordosis in standing using pelvic control reduces facet compression bilaterally.

Selecting Side of Location

The side of the back pain is the side of location of the monitoring apparatus. If the pain is central the side is not important.

Positioning Device

The device is attached to a belt positioned above the centre point of the iliac crest and the twelfth rib on the specific side.

Positioning the Patient

The wearer is shown how to stand with back to the wall and flatten the low back against the wall using the well known pelvic tilt manoeuvre, using the core trunk stabilizing muscles.

Zeroing the Device

When the low back is flat against the wall the zero is set, either by the sustained hold of the threshold setting button on the device by the patient, or by a sustained push of this button on a lead held by the clinician. The patient maintains the posture until the confirmatory 2 beeps of the device to say the position has been memorized.

Setting the Functional Movement Threshold Activity Level

The patient is instructed to place the hand between the back and the wall and push the back flat against it. The clinician then sets the functional threshold position by pushing the external switch, plugged into the device with a wire.

The patient then tests the setting by allowing the back to arch, using a forward push of the pelvis, and then stopping it by flattening the back again until the alarm ceases.

Setting the Threshold Alarm Time Delay

This time delay alarm is set at a PC interface to initially allow significant periods of pelvis forward/back arch in standing while muscle rehabilitation is undertaken and the patient strengthens.

Initially the time delay of the alarm is set at 2 minutes to allow recovery of the muscle tissue from the static contracting to hold the low back flat.

As the tissue strengthens the patient is able to maintain the back flat posture for longer and hence the time delay threshold alarm is progressively reduced, so they spend less time in the back arched/pelvis forward position.

This is the opposite of how it is used in the previous intervertebral disc problem.

Time of Recovery

This now decides how soon the patient can return to the slouched, stomach forward posture after having previously been there and having caused the threshold alarm to activate.

It effectively becomes a measure of how long the patient needs to maintain the flat back position.

Initially this can be set at a shorter time period, and steadily increased as the patients muscle rehabilitation allows them to maintain the correct position for longer.

Analysis of Data Reports for Subsequent Settings

Key analysis for this is the daily alarm behaviour display. This should register a reduction in the number of daily alarms as the core muscle control improves and the flattened back position is maintained. As the alarm frequency reduces the time period for the threshold alarm delay is reduced. This has the function of increasing the time that the flat back posture is maintained.

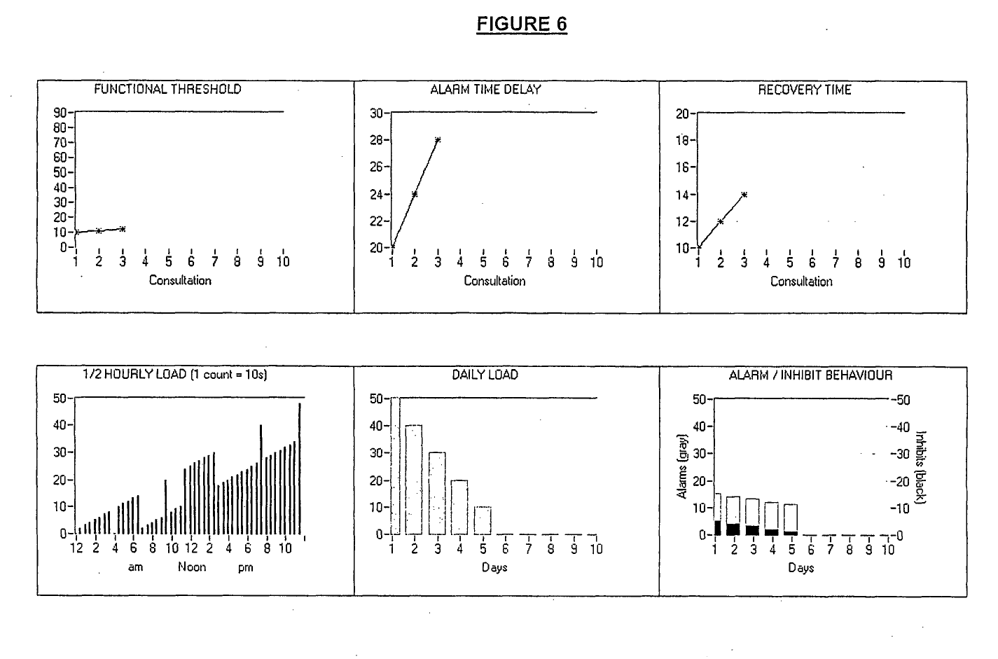

FIG. 6 shows a number of report graphs which may be displayed in conjunction with the present invention. These graphs include;

Functional Threshold

The functional angle of bending and twisting set by the clinician. When the patient exceeds this setting, the various alarm behaviours and the recording system are activated

Alarm Delay Time

The time the patient is able to exceed the functional threshold before the alarm activates

Recovery Time

The recovery following an alarm when the patient must not exceed the functional threshold. If they exceed the functional threshold during this time, the alarm sounds immediately

½Hour Loading

Displays the total time exceeding the functional threshold in real time in ½ hourly periods.

A steadily increasing level of movement beyond the threshold during the day is an indication that the patients condition is improving as long as there is corresponding reduction in pain levels by NRS for this Discogenic back pain diagnosis on consultation.

This would indicate it is appropriate to increase the functional threshold setting including functional range and time periods of load (threshold alarm time delay) while reducing the recovery time (threshold alarm recovery delay)

Conversely if the patient was showing no improvement or a worsening in pain levels with this type of loading pattern, it would be appropriate to reduce the functional threshold whilst reducing the threshold alarm time delay and increasing the threshold alarm recovery delay.

Daily Load

Displays the total amount of time exceeding the functional threshold on the previous days.

This pattern of loading over time with a fixed functional threshold shows a steadily reducing level of motion in this region instead of a progressive increase, which is the goal.

This demonstrates the patient is less mobile as the day's progress and would be of concern whether the patients pain had reduced or not in the acute phase.

At this stage it would be appropriate to change the way the device is used and set loading targets of the patient achieving the alarm going off on a regular basis.

This would include changing settings to reducing threshold recovery time alarm, and increasing the threshold alarm time delay.

It could indicate that more motivational input/education and pain relief medication may be necessary for this patient.

Alarm/Inhibit Behaviour

Displays of the number of times the alarm was activated (grey) and the number of times it was inhibited (Black) on a daily basis.

This shows how often the alarm (which is a component of threshold exceeded, time, and lack of recovery) was activated daily.

Alarms being recorded on an increasing basis with an improving patient indicate it is appropriate to increase the threshold and/or the time loading, and/or to reduce the recovery time.

If the patient is worsening then it may be appropriate to reduce the time delay alarm and increase the recovery period time based on this graph.

The alarm inhibits in black are done by the patient by pressing the threshold setting button once, and they are instructed only to perform this in a safe position (trunk weight supported by the hands)

The alarm inhibit button removes the data that activated the alarm in a safe position from the ½ hourly and the daily loading displays. This excludes data that is not loading the damaged disc region.

Aspects of the present invention have been described by way of example only and it should be appreciated that modifications and additions may be made thereto without departing from the scope thereof.

1-26. (canceled)

27. A biomechanical monitoring apparatus which includes,
a mounting means adapted to allow the apparatus to be worn by a user, and
a motion detection means adapted to detect motion of a user when the apparatus is worn by said user, and
an accumulation means adapted to indicate the extent of motion activity completed by a user since a specific time,
wherein the accumulation means is adapted to compare motion activity detected with at least one threshold activity level to indicate a time period over which this threshold activity level was exceeded.

28. A biomechanical monitoring apparatus as claimed in claim 27, said apparatus being adapted to monitor movement activity related to a user's lower back.

29. A biomechanical monitoring apparatus as claimed in claim 28, wherein a threshold activity level indicates a degree of bodily extension.

30. A biomechanical monitoring apparatus as claimed in claim 28, wherein a threshold activity level includes an indication of a degree of bodily extension and a time duration indication.

31. A biomechanical monitoring apparatus as claimed in claim 27, wherein the mounting means is adapted to mount the monitoring apparatus on a user above the user's pelvis and below the user's lowest rib at the thorax.

32. A biomechanical monitoring apparatus as claimed in claim 27, wherein the mounting means is adapted to mount the monitoring apparatus on a user below the end of the user's twelfth rib and at substantially the middle of the iliac crest of the ilium.

33. A biomechanical monitoring apparatus as claimed in claim 27, wherein the mounting means is formed by a belt adapted to encircle and engage with the body of a user.

34. A biomechanical monitoring apparatus as claimed in claim 27, wherein a motion detection means is formed from at least one accelerometer.

35. A biomechanical monitoring apparatus as claimed in claim 27, which includes an indicator means adapted to signal when a threshold activity level has been exceeded by a user.

36. A biomechanical monitoring apparatus as claimed in claim 27 wherein the accumulation means is formed from a microprocessor and a memory element.

37. A biomechanical monitoring apparatus as claimed in claim 36, wherein the accumulation means is configured to upload an accumulative indication of the extent of the motion activity completed by a user to a remote computer system.

38. A biomechanical monitoring apparatus as claimed in claim 27, which includes a user operable threshold setting element.

39. A method of using a biomechanical monitoring apparatus; said apparatus including an accumulation means adapted to indicate the extent of motion activity completed by the user; characterised by the steps of;
(i) mounting the biomechanical monitoring apparatus on a user, and
(ii) initialising the biomechanical monitoring apparatus when the user holds an initialisation stance, and
(iii) setting at least one threshold activity level for said user, and
(iv) comparing motion activity indicated by said accumulation means with at least one threshold activity level to indicate a time period over which this threshold activity level was exceeded.

40. A method of using a biomechanical monitoring apparatus as claimed in claim 39, further characterised by the additional subsequent step of;
(iv) determining the orientation of the biomechanical monitoring apparatus when mounted on the user.

41. A method of using a biomechanical monitoring apparatus as claimed in claim 39, wherein the monitoring apparatus is mounted on a user above the user's pelvis and below the user's lowest rib at the thorax.

42. A method of using a biomechanical monitoring apparatus as claimed in claim 39, wherein the biomechanical monitoring apparatus is mounted on a user below the end of the user's twelfth rib and at substantially the middle of the iliac crest of the ilium.

43. A method of using a biomechanical monitoring apparatus as claimed in claim 39, wherein a threshold activity level is set for a user during the user executing at least one hazardous motion.

44. A method of using a biomechanical monitoring apparatus as claimed in claim 39, wherein a threshold activity level is set at a position within the user executing a hazardous motion at which the user first experiences pain.

45. A method of using a biomechanical monitoring apparatus as claimed in claim 39, wherein previously accumulated indications of the extent of motion activity of a user are used to update said at least one threshold activity level for said user.

46. A method of using a biomechanical monitoring apparatus as claimed in claim 39, wherein said at least one threshold activity level set for a user is updated iteratively over time as the user follows a treatment plan.

47. A method of using a biomechanical monitoring apparatus characterised by the steps of:
(i) mounting the biomechanical apparatus on a user to allow the user to wear said apparatus, and
(ii) detecting at least one specific type of motion of the user wearing the apparatus, and
(iii) activating the accumulation means of the apparatus to provide an accumulative indication of the extent of motion activity completed by the wearer since a specific time, and
(iv) comparing motion activity indicated by the accumulation means with at least one threshold activity level to indicate a time period over which this threshold activity level was exceeded; and
(v) repeating step (iv) as above for each subsequent motion of the user detected.

48. A method of using a biomechanical monitoring apparatus characterised by the steps of:
(i) mounting the biomechanical apparatus on a user to allow the user to wear said apparatus, and
(ii) detecting at least one specific type of motion of the user wearing the apparatus, and
(iii) activating the accumulation means of the apparatus to provide an accumulative indication of the extent of motion activity completed by the wearer since a specific time, and
(iv) processing the accumulative indication of the extent of motion activity supplied by the accumulation means using a processing means, and
(v) comparing motion activity indicated by the accumulation means with at least one threshold activity level to indicate a time period over which this threshold activity level was exceeded; and
(vi) repeating steps (iii) through (v) as above for each subsequent motion of the user detected.

49. A method of using a biomechanical monitoring apparatus as claimed in claim 47, further characterised by the additional subsequent step of activating an indicator means to signal when a threshold activity level has been exceeded by the user wearing the apparatus.
